(19) United States
(12) Patent Application Publication
(10) Pub. No.: US 2006/0206144 A1
(43) Pub. Date: Sep. 14, 2006

(54) Medical instrument with actuating handle

(75) Inventor: Hannes Miersch, Hamburg (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/370,352

(22) Filed: Mar. 8, 2006

(30) Foreign Application Priority Data
Mar. 11, 2005 (DE) 10 2005 011 787.2

(51) Int. Cl. A61B 17/00

(52) U.S. Cl. 606/205

(57) ABSTRACT

A medical instrument with at least one movable element (4) on its distal end which is in actuating engagement with a proximally disposed handle (7,8), with two gripping parts (7, 8) of the handle which are articulated on one another by way of a joint (9) and have finger grips (10, 11) on their free ends, and with a ratchet lock (12, 13) between the gripping parts (7, 8) which comprises two toothed racks (12, 13) which are constructed concentrically with the joint (9) and can be brought into locked engagement with one another and of which one (12) is fixed on a first gripping part (7) and the other (13) is fixed on a pivot lever (14) pivotably mounted on the second gripping part (8) and is pressed by a spring (16) into the locked position, wherein the pivot lever (14) is lengthened beyond its bearing point (15) to form an actuating part (17) and is provided with a retaining device (19, 20; 23, 24; 25, 26) for fixing it in the unlocked pivoted position, characterised in that the retaining device (19, 20; 23, 24; 25, 26) is constructed so as to retain the pivot lever (14) in a force-locking manner relative to the second gripping part (8).

### BACKGROUND OF THE INVENTION

1. Field of the Invention

The invention relates to medical instruments.

2. Description of the Related Art

Medical instruments are used in surgery, typically in the form of shears or forceps in which the closing movement is controlled by a handle.

Such instruments can be designed, for example, for laparoscopic use, in which case they have a forceps head at the distal end of an elongate stem. The actuating engagement between the forceps head and the handle is brought about by a pull rod.

A ratchet lock, which is held in engagement by a spring, is provided between the gripping parts of the handle. After closure of the forceps, the ratchet lock maintains the engagement so that the forceps holds an object, e.g. a needle, an organ or the like, firmly without further finger pressure.

A pivot lever can be retained against a spring force in the unlocked pivoted position by a retaining device, which provides the possibility of also working with such a forceps without engagement of the ratchet lock.

A generic instrument is known from DE 93 14 581 U. In this case the retaining device is constructed as a securing lever which can be brought into securing engagement on the actuating part of the pivot lever.

However, a disadvantage of this design is the complicated actuation of the securing lever which is to be brought into and out of engagement by a separate pivoting movement, thus unnecessarily occupying the attention of the surgeon during complicated operations.

The object of the present invention is to create a generic instrument in which the retaining device can be operated more simply for securing of the pivot lever.

### BRIEF SUMMARY OF THE INVENTION

According to the invention, a retaining device is designed to retain a pivot lever in the locked position in a force-locking manner. Together with a spring which presses the pivot lever into the locked position this produces a bistable mounting of the pivot lever. Thus the pivot lever can be brought by finger actuation of its actuating part in one direction into the unlocked position and secured in a force-locking manner. In order to bring it back into the locked position it is sufficient to press its actuating part with the finger in the other direction in order to release the force-locking connection. This operation can be carried out by the surgeon intuitively and without diversion.

Upon actuation of the pivot lever a cam which pivots with it is moved past a resilient catch into one or the other pivoted position, so that it can be brought with a light pressure into the locked or unlocked position.

In the unlocked position the pivot lever is retained magnetically on a second gripping part. This magnetic hold can be released again with a light finger pressure. A construction is possible with permanent magnets on both parts or with a permanent magnet on one of the parts, the other part being made from ferromagnetic material.

The force-locking engagement between the pivot lever and the second gripping part takes place by engagement of a shaped part on one of the parts in a spring seat on the other part which retains the pivot lever in the unlocked position and can be released again with a light pressure.

The invention is illustrated by way of example and schematically in the drawings.

### BRIEF DESCRIPTION OF THE DRAWINGS

FIG. 1 shows a first embodiment of a surgical forceps,

FIG. 2 shows a detail of FIG. 1 in a second embodiment, and

FIG. 3 shows a detail of FIG. 1 in a third embodiment of the invention.

### DETAILED DESCRIPTION OF THE INVENTION

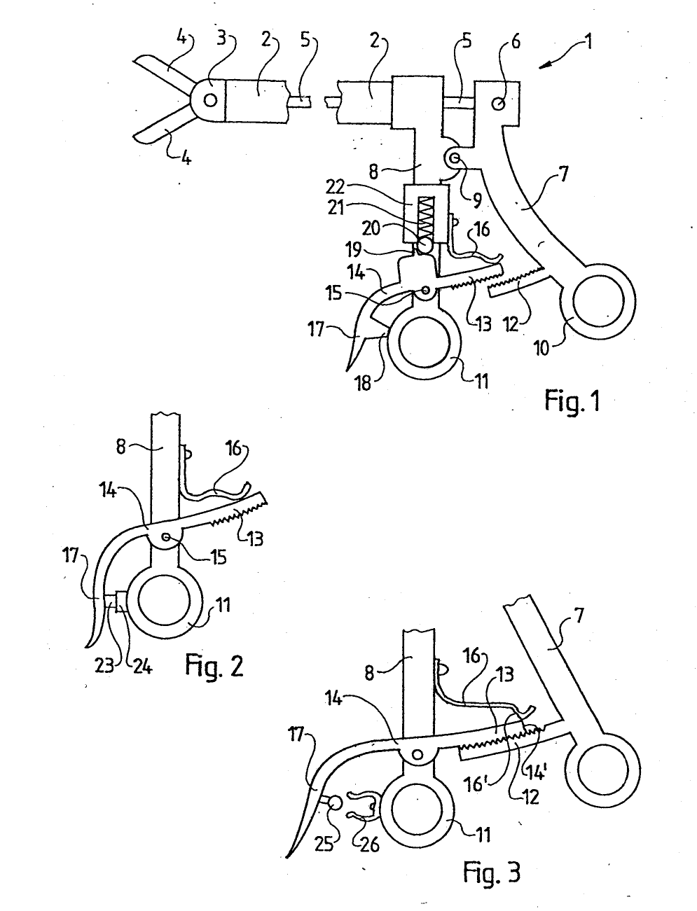

FIG. 1 shows a surgical forceps 1 with an elongate tubular stem 2 which has fixed on its distal end a forceps head 3 which can also, for example, be constructed as shears and has one or two movable arms 4. The forceps head 3 is actuated by means of a pull rod 5 which passes through the tubular stem 2.

The pull rod 5 extends beyond the proximal end of the tubular stem 2 and is mounted by means of a ball joint 6 or the like on a first gripping part 7 which forms a handle with the second gripping part which bears the tubular stem 2. The two gripping parts 7, 8 are articulated on one another by a joint 9.

Finger grips 10, 11 are situated at the free ends of the gripping parts 7 and 8 In this embodiment, the finger grips 10, 11 are constructed as finger rings, the finger grip 10 being provided for the thumb and the finger grip 11 for the index finger of the surgeon.

The illustrated surgical forceps 1 are actuated by pressing together the finger grips 10, 11 with the fingers of one hand, whereby depending upon the chosen kinematics when the finger grips 10, 11 are pressed together the arms 4 of the forceps head 3 are closed or opened.

A first toothed rack 12 extending concentrically with the joint 9 is fixed on the first gripping part 7, projecting towards the second gripping part 8. This first toothed rack forms, together with a second toothed rack 13, a ratchet lock which is constructed as one arm of a pivot lever 14 which is pivotably mounted on the second gripping part 8, at a bearing point 15. By means of a leaf spring 16 fixed on the second gripping part 8 the pivot lever 14 is biased into the locked position in which the second toothed rack 13 is retained in ratchet engagement with the first toothed rack 12.

A second arm of the pivot lever 14 is constructed as an actuating part 17 and, when the surgical forceps 1 is held with the thumb in the finger grip 10 and the index finger in the finger grip 11, can be actuated, for example, by the middle finger in order to bring the pivot lever 14 into the unlocked position shown in FIG. 1, which in the illustrated embodiment is secured by a stop 18 on the actuating part 17 against the finger grip 11.

In the unlocked position of the ratchet lock 12,13 shown in FIG. 1 the forceps 1 can be actuated freely without disruption by the ratchet lock. However, for this purpose the actuating part 17 would have to be held continuously.

In order to avoid this, a retaining device is provided which can hold the pivot lever 14 in the unlocked pivoted position shown in FIG. 1.

As FIG. 1 shows in a first embodiment, the retaining device on the pivot lever 14 has a cam 19 which pivots therewith and which in the illustrated position in the view of FIG. 1 is to the left of a catch 20 constructed as a ball which is biased by a spring 21 and mounted in a shaft part 22 which is fixed on the second gripping part 8.

With the cam 19 to the left of the catch 20 the unlocked pivoted position of the pivot lever 14 is secured, as FIG. 1 shows. In order to bring the pivot lever 14 back into the locked position it is merely necessary to move the actuating part 17 of the pivot lever 14 away from the finger grip 11 using the finger. The cam 19 passes through, under the resiliently yielding catch 20 towards the right, so that the leaf spring 16 can bring the second toothed rack 13 back into ratchet engagement with the first toothed rack 12.

FIGS. 2 and 3 show, in details of FIG. 1, alternative embodiments of the retaining device for the ratchet lock using the same reference numerals where possible.

According to FIG. 2 a permanent magnet 23, 24 is fixed respectively on the actuating part 17 and on the finger grip 11 in order to secure the pivot lever 14 in the illustrated unlocked position. In the illustrated pivoted position of the pivot lever 14 the permanent magnets 23 and 24 are in magnetic engagement and retain the pivot lever 14 in the unlocked position against the force of the leaf spring 16. If the magnetic engagement is released by finger pressure on the actuating part 17 in the direction away from the finger grip 11, then the pivot lever 14 pivots into the locked position. The operation of the retaining device, shown in FIG. 2, is exactly the same as in that of FIG. 1. A construction is possible with permanent magnets on both parts or with a permanent magnet on one of the parts, the other part being made from ferromagnetic material.

FIG. 3 shows a retaining device which is of similar construction to that of FIG. 2. However, in this case instead of the magnets 23, 24, a shaped part 25 which in this embodiment is spherical is fixed on the actuating part 17 and a spring seat 26 formed with a leaf spring is fixed on the finger grip 11. If the pivot lever 14 is moved in the direction of the unlocked position, that is to say the actuating part is moved towards the handle 1, then the shaped part 25 enters the spring seat 26 and is retained there by spring force. By movement of the actuating part 17 in the direction of the locked position shown in FIG. 3 the shaped part 3 is released by overcoming the spring force of the spring seat 26. Also in this construction of the retaining device 25, 26 the operation is again the same as in the embodiments of FIGS. 1 and 2.

As an alternative to the embodiment of FIG. 3, the leaf spring 16 can be lengthened somewhat in such a way that upon movement of the pivot lever 14 into the unlocked position it engages with the angled end 16′ behind the end 14′ of the pivot lever 14 and retains it in the unlocked position. For locking, the lever 14 is disengaged from the points 16′, 14′ by action of a light force. Also, in this way, the pivot lever 14 can be bistably secured.

As an alternative to the embodiment of FIG. 1, the spring 21, which can also for example be constructed as a leaf spring, can be fixed at its upper end on the gripping part 8. The shaft part 22 is omitted. The lower end of the spring is fixed at the location of the cam 19 on the pivot lever 14. This also results in a bistable mounting of the pivot lever 14 which securely holds the pivot lever in the locked or unlocked position.

1. A medical instrument including at least one movable element on its distal end which is in actuating engagement with a proximally disposed handle comprising two handle members, which are pivotally connected together by means of a joint and have finger grips on their free ends, and a ratchet lock between the handle members, which comprises two arcuate toothed racks which are concentric with the joint and can be selectively moved into locked engagement with one another and of which one is connected to the first handle member and the other is carried by a pivotal lever pivotably mounted on the second handle member and is urged by a spring into the locked position, wherein the pivotal lever is extended beyond its pivot point to form an actuating member and is provided with a retaining device for securing it in the unlocked position, the retaining device being so constructed and arranged in a force-locking manner that the pivotal lever may be released from the locked position merely by the application of a force to the actuating member.

2. An instrument as claimed in claim 1, in which a cam is connected to the pivotal lever and pivots with it and, in the unlocked position of the pivotal lever, engages behind a catch resiliently mounted on the second handle member.

3. An instrument as claimed in claim 1, in which a permanent magnet arranged to hold the pivotal lever in the unlocked position is disposed on at least one of the pivotal lever and the second handle member.

4. An instrument as claimed in claim 1, in which a shaped member is connected to the pivotal lever and in the unlocked position of the pivotal lever, engages resiliently in a spring seat on the second handle memberor.

5. An instrument as claimed in claim 1, in which a shaped member is connected to the second handle member and in the unlocked position of the pivotal lever, engages resiliently in a spring seat on the pivotal lever.
